(19) United States
(12) Patent Application Publication
(10) Pub. No.: US 2008/0039834 A1
(43) Pub. Date: Feb. 14, 2008

(54) Electrosurgical probe

(76) Inventor: Dale Victor MacKay, Carlton North Victoria (AU)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 11/891,343

(22) Filed: Aug. 10, 2007

Related U.S. Application Data
(63) Continuation-in-part of application No. PCT/AU2006/000159, filed on Feb. 10, 2006.

(30) Foreign Application Priority Data
Feb. 11, 2005 (AU) 2005900641
Feb. 16, 2005 (AU) 2005900726

(51) Int. Cl. A61B 18/14

(52) U.S. Cl. 606/46; 600/104

(57) ABSTRACT

A probe (100) for use in electrosurgery includes a lumen (101) having a proximal end (101a) and a distal opening (101b). There is an electrode (103) within the lumen which has a proximal end (103a) connectable to a high frequency current source (105) and a discharge end (103b) recessed within the lumen. The lumen includes a gas outlet (107) in a distal region of the lumen for releasing gas pressure. During use, when the distal opening (101b) of the lumen (101) contacts target tissue while the electrode is activated, gas pressure within the lumen is released through the gas outlet (107) thus avoiding gas embolism. A probe attachment (300) is also provided.

### FIELD OF THE INVENTION

The present invention relates to electrosurgery and to probes for use with electrosurgical devices. It relates particularly but not exclusively to probes and probe attachments for use in endoscopic electrosurgery.

### BACKGROUND TO THE INVENTION

Electrosurgery uses high frequency energy to cauterise, burn or ablate tissue whilst minimising bleeding. Electrosurgery can be practiced using bipolar or monopolar modality. In a bipolar mode, electrosurgical equipment passes a current between two electrodes resulting in an arc between the electrodes. Tissue located between the electrodes is vaporised or cauterised by heat generated by the electrical discharge thereby creating an electrosurgical cut or cauterisation of the tissue.

In monopolar modality, the patient forms part of the electric circuit. Thus high frequency current applied by a single discharge electrode travels through the patient's body to a return electrode attached elsewhere on the patent which is in turn connected to ground. In an isolated circuit, the patient return electrode is connected to the electrosurgical generator unit thus completing the electric circuit with a floating electrical potential between the discharge and the return electrodes. This reduces the risk of accidental patient burns at non-surgical sites that can be caused by the applied current seeking an alternate path to ground.

A range of tissue effects can be created using electrosurgical devices. These include cutting, fulguration, desiccation, coagulation and ablation. In cutting, a lower voltage, high duty cycle RF (radio frequency) current is focussed at the site creating intense heat. This causes vaporisation of tissue accompanied by hemostasis in which bleeding at the site is minimised or prevented. Fulguration is usually performed with a modulated power source at a reduced duty cycle such that the high frequency energy is applied to the site in short bursts. This dehydrates, coagulates and chars the tissue over a larger area, also sealing the tissue. For desiccation, the electrode is usually in direct contact with the target tissue thereby reducing the current concentration. Thus, less heat is generated causing the cells to dehydrate and form a coagulum, rather than vaporising as is the case in cutting. In each mode, different effects can be produced by varying the waveform used to energise the discharge electrode and by altering the distance between the discharge electrode and the target tissue.

Control of the electrosurgical energy applied to the target tissue has been improved by introducing a gas stream into the discharge path of the electrode. In gas enhanced electrosurgery, an easily ionised inert gas such as argon or helium is introduced to the surgical site and used to form a shroud around the discharge electrode. This has the effect of moderating the electrical discharge. The gas also blankets the surgical site thereby suppressing charring of tissue in the area. Use of ionisable gas in this way is also thought to reduce heating of the discharge electrode thus inhibiting electrode destruction and eschar build up.

The inert gas provides a more conductive medium than oxygen and other gases which would ordinarily exist around the discharge electrode. Thus, current discharged from the electrode seeks the path of least resistance, ionising the argon stream and arcing to the tissue bed. This results in better directional delivery of the RF current to the target tissue and causes less damage than conventional RF current. Moreover, since the ionisable gas displaces oxygen in the discharge path there is less carbonisation and therefore less eschar build up, accompanied by decreased smoke plume.

Advantageously, the level of hemostasis created using electrosurgery reduces the amount of trauma caused to the tissue during the procedure. This facilitates faster wound healing with reduced propensity for complications arising from infection. In the early 1990's the concept of utilising electrosurgical techniques through a flexible endoscope was investigated. Flexible endoscopes are used to view internal organs and tissues of a patient. Procedures using flexible endoscopes are usually performed by gastroenterologists, surgeons and respiratory physicians to explore and treat organs including the gastrointestinal tract and bowels, bronchioles and lungs, and joints and other tissues which can be viewed laparoscopically.

Endoscopic electrosurgery has been used to achieve dissection and hemostasis. To treat lesions on the mucosal wall of the gut, for example, the surgeon holds the discharge electrode tip at a distance from the target tissue and activates the electrode to generate an arc to the tissue. Hence the desired tissue coagulation or fulguration is achieved. There is, however, an associated risk of burning too deeply into the mucosal wall because it is harder to control the electrosurgical discharge endoscopically than it is in open surgery procedures. Delivering the RF current through a stream of inert gas such as argon improves control over delivery of the RF energy. Therefore gas enhanced electrosurgery is common in endoscopic procedures.

However, such procedures are not performed without risk, and the known electrosurgical probes are not well suited for use in the gastrointestinal tract, for example, because during most endoscopic procedures the patient is heavily sedated and can move around due to breathing or discomfort causing the target tissue to move during treatment. This increases the likelihood of the probe tip coming into contact with the mucosal wall and during delivery of gas and activation of the electrode this can cause perforation of the mucosal wall and gas embolism in the target tissue. Accordingly, use of these probes in endoscopic procedures is potentially dangerous. When these probes are used endoscopically the surgeon's technique is vital to ensure that the probe does not make contact with the mucosal wall while the electrode is activated.

Experienced surgeons may develop a feel for the patient's movements and move the probe away from the tissue surface and/or de-activate the electrode (e.g. by releasing a foot-operated pedal) in anticipation of the probe tip contacting the mucosal wall. However, this is not a fail-safe approach and even the most experienced surgeons make occasional contact between the activated electrode and the tissue surface putting the patient at risk of gas embolism. It would be desirable to provide a probe which is safe for use in endoscopic electrosurgery.

It is an object of the present invention to overcome or ameliorate one or more of the disadvantages of the prior art, or at least to provide a useful alternative.

The discussion of the background to the invention included herein including reference to documents, acts, materials, devices, articles and the like is intended to explain the context of the present invention. This is not to be taken as an admission or a suggestion that any of the material referred to was published, known or part of the common general knowledge in Australia as at the priority date of any of the claims.

### SUMMARY OF THE INVENTION

In a first aspect of the present invention there is provided a probe for use in electrosurgery, the probe including:

(a) a lumen having a proximal end and a distal opening configured for contacting target tissue to achieve a treatment effect;

(b) an electrode within the lumen, the electrode having a proximal end connectable to a high frequency current source and a discharge end recessed within the distal end of the lumen; and

(c) a gas outlet in a distal region of the lumen for relieving gas pressure;

wherein the distal opening of the lumen contacts target tissue during use while the electrode is activated, and excess gas pressure within the lumen is relieved through the gas outlet.

The distal opening may be shaped to create a desired treatment effect in the target tissue. Preferably, the distal opening is oblique to facilitate treatment of a larger area of target tissue. This also enables a surgeon to have better directional control of the high frequency current when applied to target tissue endoscopically. When the distal opening is oblique it is preferred that the gas outlet is positioned such that the likelihood of both the distal opening and the gas outlet contacting or becoming occluded by the target tissue simultaneously is minimised. Thus, the gas outlet is preferably located in a wall of the lumen substantially opposing the distal opening. The gas outlet facilitates dynamic relief of excess gas pressure within the lumen during use of the device. This creates an equilibrium of pressure across the gas outlet enabling electrons to ionise gas within the lumen thereby ablating target tissue at the tissue-gas interface, within the confines of the walls defining the distal opening.

Whilst the gas outlet may be provided anywhere in the distal region of the lumen which permits release of excess gas pressure within the lumen, it is preferably provided adjacent the discharge end of the electrode and may be located just distal of the discharge end of the electrode. Whilst it may be sufficient to provide one gas outlet, it is possible that more than one gas outlet may be provided to release pressure created by gas within the lumen thereby enhancing dynamic pressure relief. Where more than one gas outlet is provided, the plurality of outlets may be smaller to provide a similar sum total gas release as a single, larger gas outlet. The outlet may take any suitable shape including round or oval, for example. Alternatively, the gas outlet may be provided by a series of holes or slots.

Preferably, a distal region of the lumen includes a transparent portion. The transparent portion may be provided in the form of a transparent window, or the distal region of the probe may be formed entirely of a transparent material. Preferably the transparent portion is ceramic, although other suitable insulating materials with heat resistant properties may be used.

In one embodiment, the distal opening has a low coefficient of friction coating for suppressing eschar build up and also to prevent tissue, blood and mucous from adhering during use. The distal region may also include a marking to indicate the orientation of the distal opening when in use. In one embodiment, the probe further includes pivot means for enabling the distal opening to maintain contact with the target tissue during movement of the probe or target tissue. Preferably, the gas within the lumen is argon gas although helium or other ionisable gases may be used.

In a preferred embodiment, a distal portion of the electrode is tungsten with a highly conductive coating at its working tip. One suitable coating is silver. The probe is suitable for use within a working channel of a flexible endoscope. Such endoscopes include devices adapted for use in the gastrointestinal tract and/or respiratory system, and devices used in laparoscopic surgery.

Target tissue is treated using the probe by contacting the distal opening to the target tissue during activation of the electrode. In use, the distal opening may also be drawn along the target tissue surface during activation of the electrode to achieve ablation of a larger region of tissue. This may incorporate a sweeping motion. Excess gas pressure is relieved by escaping through the gas outlet thereby facilitating dynamic pressure relief during use.

According to another aspect of the present invention, there is provided an attachment for an electrosurgical probe having an electrode, the attachment including:

(a) a lumen having a proximal end connectable to the probe and a distal opening being a working tip; and

(b) a gas outlet in the lumen for releasing gas pressure;

such that when the attachment is coupled to the probe, the electrode is recessed within the lumen so as to preclude direct contact with target tissue, and wherein the working tip contacts the target tissue during activation of the electrode to achieve a treatment effect, with excess gas pressure within the lumen is relieved through the gas outlet.

The gas outlet may be provided just distal of the discharge electrode. The distal opening may be shaped to create a desired treatment effect in the target tissue. Preferably, the distal opening is oblique. Preferably, the gas outlet is positioned so as to minimise the risk of the gas outlet and distal opening contacting the tissue simultaneously. Thus, the gas outlet is preferably provided in a wall of the lumen substantially opposing the oblique distal opening. The gas outlet provides dynamic pressure relief, during use of the probe to, for example, ablate tissue.

In one embodiment, the probe attachment includes transparent window portion although the attachment itself may be transparent. The probe attachment may be manufactured from a ceramic material, or any other insulator which can withstand high temperatures without deforming or being otherwise affected. In one embodiment the distal opening has a low coefficient of friction coating to suppress eschar build up during use and to prevent blood, mucous and tissue from adhering to the working tip.

In one embodiment, the attachment includes a marking on the lumen to indicate the orientation of the distal opening when in use. The attachment may also include pivot means for enabling the distal opening to maintain contact with the target tissue during movement of the probe or target tissue. The pivot means may take any suitable form such as a swivel or other rotatable connection.

Preferably, the probe attachment is suitable for use with a probe adapted for use within a working channel of a flexible endoscope. Such probe may be used to treat the gastrointestinal tract or respiratory system, for example.

According to another of its aspects, the present invention provides an endoscopic electrosurgery probe including a flexible lumen having a proximal end connectable to a source of ionisable gas and a distal opening, an electrode having a proximal end attachable to a high frequency current source and a discharge tip recessed within a distal region of the lumen so as to preclude direct contact with target tissue, the lumen also having a gas outlet near the electrode discharge tip, wherein during use, the distal opening contacts target tissue while the probe is activated by shrouding the electrode in ionisable gas and applying high frequency current to the electrode to create a treatment effect in the target tissue at the gas-tissue interface, and wherein excess gas pressure within the lumen is released through the gas outlet.

In another of its aspects, the present invention provides a probe for use in electrosurgery, the probe including:

(a) a lumen having a proximal end and a distal opening;

(b) an electrode within the lumen, the electrode having a proximal end connectable to a high frequency current source and a discharge end recessed within the lumen; and

(c) a gas outlet in a distal region of the lumen for releasing gas pressure;

wherein during use, when the distal opening of the lumen contacts target tissue while the electrode is activated, gas pressure within the lumen is released through the gas outlet thus avoiding gas embolism.

### BRIEF DESCRIPTION OF THE DRAWINGS

The present invention will now be described in greater detail with reference to the accompanying drawings. It is to be understood that the particularity of the accompanying drawings does not supersede the generality of the preceding description of the invention.

FIG. 1 illustrates part of a probe according to an embodiment of the invention.

FIG. 2 illustrates the tip of the probe of FIG. 1 in use, and having a transparent distal region.

FIG. 3 illustrates a probe attachment according to another embodiment of the invention.

FIG. 4 is a top view of a probe according to another embodiment of the present invention, having pivot means and a marking to indicate the orientation of the distal opening.

FIG. 5 is an exploded view of the probe of FIG. 4 showing the pivot means.

### DETAILED DESCRIPTION

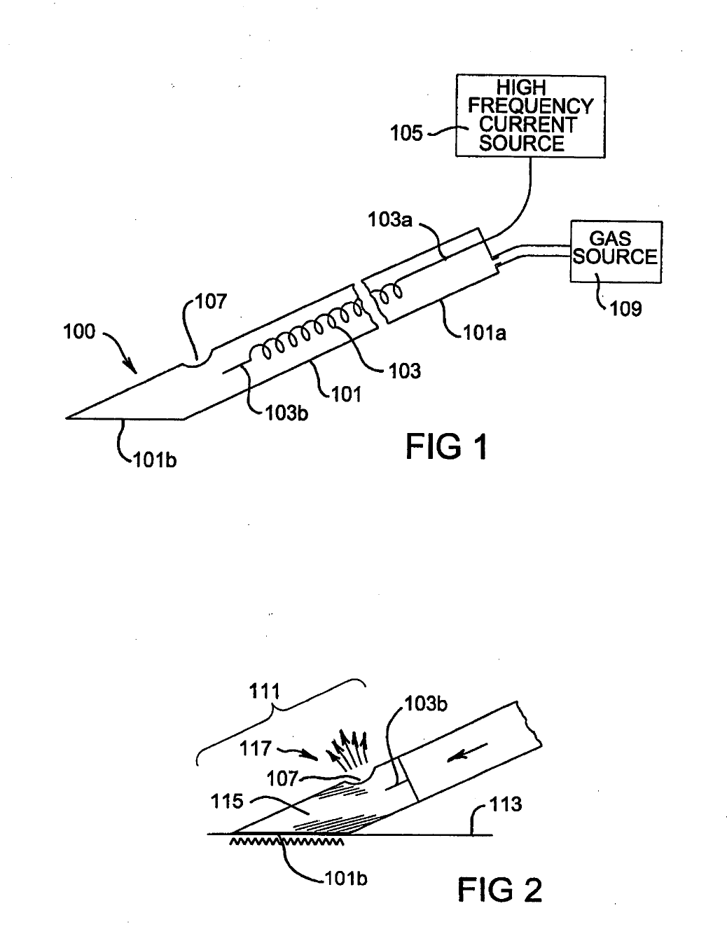

Referring firstly to FIG. 1, a probe indicated generally at 100 is shown for use in electrosurgery according to an embodiment of the invention. A lumen 101 has a proximal end 101a and a distal opening 101b. The break in the lumen between the proximal and distal ends indicates that the lumen is of variable length. An electrode 103 is provided within the lumen. The electrode has a proximal end 103a connectable to a high frequency current source 105, and a discharge end 103b recessed within the lumen distally. The discharge end of the electrode is recessed within the lumen so as to preclude direct contact between the electrode tip and the target tissue. A gas outlet 107 is provided in a distal region of the lumen for relieving excess gas pressure during use of the probe. During use, the distal opening 101b of the lumen contacts target tissue while the electrode 103 is activated with high frequency current from source 105 ionising gas within the lumen and creating a treatment effect, i.e. ablation, at the gas-tissue interface. Excess gas pressure within the lumen is released through the gas outlet 107 providing dynamic pressure control and enabling a treatment effect to occur at the tissue-gas interface, meanwhile avoiding the tendency to develop a gas embolism in the target tissue.

The probe 100 is preferably a long flexible tube connectable to a base unit which includes a high frequency current source 105. The current source may operate at radio frequency (RF) for example, although other high frequencies which may not be considered to be within the RF range may also be suitable. Preferably, the base unit also has a source of ionisable gas 109. Suitable base units include electrosurgical generator units and argon delivery systems of the kind manufactured by ConMed, Valleylab, Erbe and Soring. One particularly suitable type of base unit for use with the inventive probe is the ConMed ABC although other existing units may be equally suitable.

Connection of the proximal end of the lumen with a gas source 109 enables a stream of gas to flow through the lumen and exit through the distal opening. Upon activation of the electrode the RF current flows through and ionises the gas, utilizing it as a conduit to deliver the energy to the target tissue without direct contact with the electrode tip. Preferably, the gas is an inert ionisable gas such as argon, although other gases including helium may be used.

Electrode 103 extends inside lumen 101 and terminates before distal opening 101 b to preclude direct contact with the target tissue. Preferably, the electrode is a flexible wire having a tungsten tip at its discharge end, although the entire electrode may be made from tungsten. Tungsten provides an effective material from which an arc of RF energy can be discharged due to its high melting temperature and heat resistant properties. The working tip of the electrode may be coated with a highly conductive material. One suitable conductive material is silver. Silver is about 40% more conductive than other elements which are used to manufacture electrodes. Coating the discharge tip of the electrode with silver creates a highly conductive interface between the tungsten electrode and the ionisable gas, thereby assisting in initiation of current arcing and ionisation of the gas.

In prior art electrosurgical probes, RF current from the electrode ionises the gas shrouding the electrode creating an arc to the target tissue. It is the arc of RF energy which produces the desired ablation and haemostasis effects. Using these probes, ablation of target tissues is not sharply focussed because the stream of ionisable gas spreads and disperses as it exits the probe tip, forming a blanket over the target tissue. These probes are intended for use in a non-contact mode. That is, with a gap between the electrode and the underlying tissue so that an arc can form across the gap and ablate the tissue.

This does not create difficulty in most electrosurgical procedures where the patient is anaesthetised and the target tissue is motionless and accessible. However, maintaining the requisite arc-inducing gap between the electrode and the target tissue is more challenging in endoscopic procedures. In endoscopic electrosurgery performed in the gastrointestinal (GI) tract, for example, complications arise because the patient is usually mildly sedated and often moves during the procedure due to breathing, discomfort and internal bodily functions. This creates difficulties for the surgeon because the tissue to be ablated becomes a moving target.

Thus it can be difficult to maintain a gap between the probe and the target tissue while the electrode is activated, and the tip of the probe can inadvertently become buried into the tissue wall. Contact between the tissue wall and the probe can cause serious complications during activation of the electrode as the tissue wall can become perforated by the gas stream exiting the probe. Contact between the tissue wall and the probe can also create a potentially life threatening situation as the risk of embolism developing is significant. It is considered a contraindication to activate these prior art probes in contact-mode. It follows that it is a contraindication to use the prior art probes endoscopically in gastroscopy, colonoscopy, bronchoscopy and other procedures where the likelihood of contact between the probe and the target tissue is high.

The present invention addresses this problem by providing a probe 100 which is suitable for use in contact with the target tissue. This is due to the dynamic equilibrium lumen created by the provision of gas outlet 107 which provides pressure relief, enabling excess gas pressure to escape from the lumen 101 while the distal opening 101b is in contact with the target tissue during activation of the electrode 103 as shown in FIG. 2. Instead of the activated probe pumping gas into the mucosal wall, gas pressure within the lumen is relieved through the gas outlet 107 removing the risk of perforation by gas pressure and gas embolism. This provides control over the gas pressure inside the distal lumen and enables electrons released from the electrode tip to work at the tissue interface achieving a controlled treatment effect.

The gas outlet must be sufficiently large to allow gas to exit easily while the distal opening is in contact with the target tissue, so as to relieve excess gas pressure and thereby maintain a dynamic equilibrium of pressure across the gas outlet. For most lumens, approximately 1 mm in diameter has been found adequate for the gas outlet, as have diameters of 1.1 mm, 1.2 mm, 1.3 mm and 1.4 mm for a lumen of approximately 1 mm internal diameter. Larger and smaller diameter outlets are also contemplated. Alternatively, the gas outlet may be provided by multiple smaller holes or openings which provide an equivalent sum total in outlet opening area.

The gas outlet permits activation of the electrode and use of the probe with the distal opening 101b of the lumen in contact with or buried in the target tissue. Accordingly, during use the distal opening of the lumen may be rested on the target tissue allowing accurate, controlled ablation. Since the distal opening is in contact with the tissue, the RF energy is confined by the lumen walls and arcing to adjacent tissues and structures is minimised. This results in less indiscriminate exposure of the surrounding tissue to the applied RF current than the prior art probes.

The gas outlet 107 may be located slightly distal of the electrode 103, with the broadest region of the opening between the discharge end 103b of the electrode and the distal opening 101b of the lumen. Advantageously, the outlet can also be used as a washing port for cleaning the electrode and the distal region of the lumen to dislodge and remove blood, mucous and debris which may build up during use of the probe. The lumen can be cleaned by syringing the distal region through the gas outlet 107 with saline, water or a cleaning fluid. Alternatively, the distal region of the lumen can be cleaned by use of a stiletto inserted through the gas outlet to dislodge any debris or tissue inside the lumen.

Preferably, the lumen has an external diameter of less than about 2.4 mm and can be inserted into the working channel of a flexible endoscope of the kind routinely used in gastroscopy, colonoscopy and bronchoscopy procedures. In use, the distal end of the lumen protrudes beyond the distal end of the working channel, outside the endoscope.

FIG. 2 illustrates the working end of a probe in use, according to an embodiment of the invention. Inside the distal region 111 of the lumen, argon gas 115 shrouds the electrode 103 providing a path of least resistance for the RF current to flow from the discharge end of the electrode 103b to the target tissue 113. Electrons complete the electrical circuit by returning to the patient return electrode located elsewhere on the patient. Because the probe is suitable for use with the distal opening 101b in contact with the target tissue, the applied current is confined by the lumen walls and does not spread over the target tissue to the surrounding areas.

The ionisable gas can be accurately and directionally controlled to treat a specific target site during activation of the electrode, with the outlet 107 providing an escape route for excess gas pressure 117 during treatment. Release of gas pressure through the gas outlet prevents a build up of pressure on the mucosal wall which occurs during use of the prior art probes. This facilitates improved control over the gas pressure present at the tissue interface and alleviates the potential for the inert gas flow to perforate the tissue wall, enabling the surgeon to perform ablation of tissues without the concerns of tissue contact associated with prior art probes. The target tissue is ablated with precision and safety without risk of accidentally causing gas embolism.

The distal opening may be any suitable shape. However, in a preferred embodiment, the distal opening 101b is an oblique opening of the kind illustrated in the attached drawings. This has been found to be ideal particularly for endoscopic procedures because the surgeon is able to direct the RF energy at the tissue wall with the lumen resting along the tract (e.g. the mucosal wall). The orientation of the distal opening can be controlled for precise delivery of RF current and ablation. Also, the oblique opening permits a greater surface area of contact when directing RF energy from the electrode. This can, where desired, produce shallower ablation which is useful for surgeons working on areas of the tract that are thin walled and particularly prone to perforation, such as the Cecum.

Because the distance between the discharge electrode and the tissue remains substantially constant when the distal opening is in contact with the tissue during treatment, more consistent ablation depths can be achieved. The distal opening may be drawn along the surface of the target tissue during activation of the electrode, enabling a larger area of tissue to be treated with a substantially consistent delivery of RF energy. A sweeping motion may be used along the tissue wall and this may further assist with clearing debris from the distal region of the lumen during use. Whilst the present invention is suitable for use while maintaining contact between the distal opening and the target tissue, it may also be used with a gap between the distal opening and the target tissue.

In a particularly preferred embodiment, a portion of the distal region of the lumen is transparent. The transparent portion may be in the form of a transparent window. In a particularly preferred embodiment, the entire distal region 111 of the lumen is transparent, as illustrated in FIG. 2. This enables the surgeon to view the discharge end 103b of the electrode and monitor its condition. The surgeon can also view the inside of the distal region of the lumen ensure that effective ionisation is taking place and that the lumen is clear of blood, mucous and debris. The transparent portion also allows the surgeon to view the target tissue and the electrode during operation, and direct the argon beam at the area of tissue to be treated very accurately, and substantially without adversely affecting the nearby tissue.

The transparent portion also enables the surgeon to ascertain quickly whether the probe tip including the distal opening (and gas outlet) is clear of blood, mucous and debris. This is advantageous because debris on the electrode inhibits effective ionisation of the gas. Utilising the transparent tip, the surgeon can monitor the condition of the electrode while it is in use thereby avoiding activation without ionisation, resulting in preservation of the discharge end of the electrode. This also reduces the severe pain and discomfort caused by the prior art probes which can cause distension of the patient's gastrointestinal tract (e.g. abdomen or colon) when gas is not effectively ionised. Further, it eliminates the time wastage associated with treatment using the prior art probes which must be stopped periodically to inspect the probe and ensure that there is no debris or eschar build up on the electrode.

Preferably, the transparent portion is made from a ceramic insulating material, although other insulating materials which have high melting temperatures may be used. In a particularly preferred embodiment, the distal opening 101b is coated with a low coefficient of friction substance such as Teflon® to suppress eschar build up on the tip. A Teflon® (or other low coefficient of friction) coating also reduces the propensity for tissue to adhere to the distal opening during operation, thereby reducing inadvertent tissue damage resulting from use of a probe tip roughened by adhered tissue or the like. This is particularly valuable where a sweeping motion is being used to treat a larger region of target tissue.

In one arrangement, the distal region of the probe includes a marker to indicate the orientation of the distal opening. In the example illustrated in FIG. 4 and FIG. 5, the marker is a line 430 which extends from the furthest point of the distal opening 410b, through the gas outlet 407 and terminates just proximal of the gas outlet. Generally, this side of the probe is in view by the surgeon operating the endoscope while using the probe with the distal opening in contact with the target tissue.

In one embodiment, the probe includes pivot means for rotating the distal opening relative to the lumen, to maintain contact between the target tissue and the distal opening during movement of the probe. The pivot means may take any suitable form and in one embodiment is a swivel or other rotatable coupling with which the distal opening rotates independently of the probe body. This enables the surgeon to align or position the distal opening by resting it on the target tissue and allowing the end of the probe to swivel into the correct position to interface with the tissue without having to manipulate the probe.

One example of a pivot means is illustrated in FIG. 4 and FIG. 5 although it is to be understood that other pivot means can be incorporated as would be known to the person skilled in the art. In FIG. 4 and FIG. 5, swivel coupling 440 has, on one side, a neck 442 which terminates in a flange 441. On the other side, a groove 443 (shown in broken lines) is provided on the inside of the coupling to receive the flange 441 which freely rotates in clockwise and counter-clockwise directions.

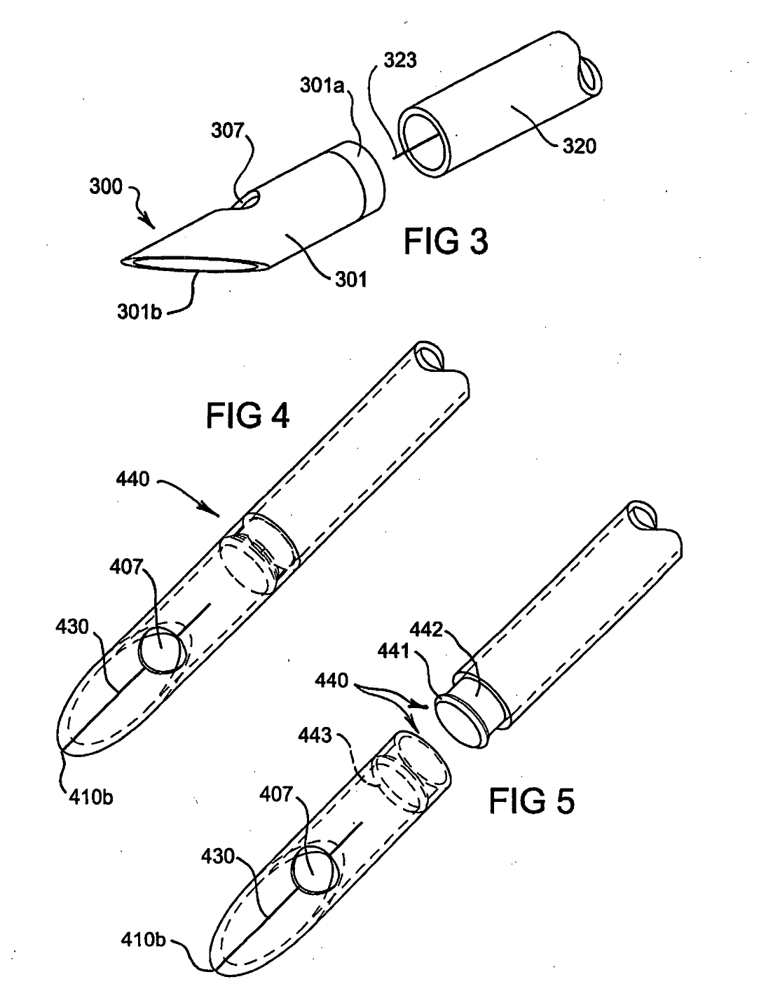

Now referring to FIG. 3, there is shown a probe attachment 300 for an electrosurgical probe 320 having an electrode 323. The probe attachment has a lumen 301 having a proximal end 301a connectable to the electrosurgical probe and a distal opening 301b being a working tip. Preferably the electrosurgical probe is a gas enhanced probe which, during operation, shrouds electrode 323 with an inert ionisable gas such as argon or helium.

The probe attachment has a gas outlet 307 which relieves excess gas pressure during use, in the same way that gas outlet 107 in probe 100 relieves excess gas pressure. The probe attachment is configured such that, when attached to probe 320, the discharge end of electrode 323 is recessed within the lumen. In this manner, when the electrode is activated an arc forms inside the probe attachment between the electrode and the target tissue. When the working tip contacts the tissue, upon activation of the electrode excess gas pressure within the lumen is released through the gas outlet. This gives the surgeon control over the gas pressure which is present at the probe tip and meanwhile, avoids perforating the tissue and also significantly reduces the risk of gas embolism.

In a manner similar to probe 100, the distal opening 301b of probe attachment 300 may take any shape, but is preferably shaped to create a desired treatment effect in the target tissue. In a preferred embodiment, the distal opening is an oblique opening, formed by terminating the lumen on an angle as illustrated in FIG. 3. This produces an increased treatment area and aids in directing the RF energy from the distal opening to the tissue mucosa when used endoscopically such as in the upper or lower gastrointestinal tracts. The treatment area can be varied by varying the angle at which the distal opening is formed. The greater the oblique opening, the larger the working. This can be advantageous when endeavouring to ablate a larger surface area of tissue such as Barrett's oesophagus.

When the opening is oblique it is preferred that the gas outlet is located such that there is very little chance of both the distal opening and the gas outlet contacting or becoming buried in the target tissue simultaneously, even when the device is used endoscopically. Accordingly, the gas outlet is preferably provided in a wall of the lumen substantially opposing the oblique distal tip. By arranging the distal opening 301b and gas outlet 307 in this way, the risk of tissue perforation or gas embolism is minimised. Preferably the outlet is provided just distal of the discharge electrode.

The probe attachment 300 may include a transparent portion. This may be in the form of a window through which a surgeon can view the interior of the probe attachment and electrode tip using an endoscope camera. In a preferred embodiment, substantially all of the probe attachment is transparent. This provides the same advantages as the transparent distal region 111 of the probe 100 described previously.

The probe attachment may be made from any sufficiently robust and heat resistant material with insulating properties. Such materials include ceramics, although various plastics and composite materials may be found suitable. Preferably, the distal opening 310b has or is coated with a low coefficient of friction material such as Teflon® to suppress eschar build up and to prevent tissue, blood and mucous from adhering to the distal opening and impeding gas flow and effective treatment using the probe with the attachment.

In a manner similar to probe 100, probe attachment 300 may also include a marking on the lumen to indicate the orientation and position of the distal opening when in use. The marking may be of the kind illustrated in FIGS. 4 and 5, or take another form which is meaningful to the surgeon.

Proximal end 301a of the probe attachment includes fastening means (not shown) to fasten probe attachment 300 to the existing electrosurgical probe 320. The fastening means may take any suitable form as is known in the art. One example utilises a spring-loaded sleeve and pin type connector, commonly called a bayonet mount. Another method may utilise a close tolerance collar and stem assembly having a snap ring or locking collar to hold the probe attachment in place. Alternatively, a screw-type fitting may be used where one of the proximal end 301a of the probe attachment and the end of the electrosurgical probe 320 have an inside or outside thread configured to engage the outside or inside thread of the other piece.

In one embodiment, the probe attachment includes pivot means akin to the pivot means 440 illustrated in FIG. 4. The pivot means may be incorporated into the fastening means, or may be provided in addition thereto. The pivot means may be in the form of a swivel or other rotatable coupling which permits the distal opening to rotate independently of the body of the probe lumen. The pivot means enables a physician to maintain contact between the distal opening of the probe attachment and the target tissue during movement of either the probe or target tissue.

In a manner similar to the probe 100, probe attachment is preferably adapted for use with an electrosurgical probe which is suitable for use when inserted in the working channel a flexible endoscope. Such endoscope may of the type used to treat the gastrointestinal tract or respiratory system.

The present invention in its various embodiments enables controlled contact-mode ablation of the target tissue, even when used endoscopically. Control is enhanced by provision of a Dynamic Equilibrium Lumen (DEL) in which gas pressure is stabilised by relief of excess gas pressure by a gas outlet. Further, the surgeon's ability to view the working tip of the device when distal region is transparent, or a transparent window is provided in the distal region of the lumen enhances use of the probe. This visualisation also enables the surgeon to recognise when the working tip is affected by blood, mucous or debris collected during a procedure and can prompt immediate remedial action by the surgeon cleaning the tip of the device. Cleaning is also facilitated by the gas outlet. This enables continued effective ionisation of the inert gas within the lumen, and effective ablation of the target tissue with reduced carbonisation and smoke plume.

Thus the present invention provides a novel probe and probe attachment for use in electrosurgery. The design enables controlled directional contact between the distal opening of the probe/probe attachment and the target tissue during ablation which renders it safer for use in endoscopic procedures. When using the present invention for endoscopic surgery, a surgeon does not need to apply the same degree of caution as is necessary when using the prior art probes since the risk of embolism is reduced significantly.

When used in contact with the target tissue, the present invention also limits indiscriminate spread of high frequency current to the surrounding tissues. Confining the high frequency energy to the tissue exposed by the distal opening results in less energy loss to the surrounding tissues so the probe can be used at significantly lower power settings. For example, using the present invention, gentle ablation may be achieved using a power setting as low as 10 Watts compared with 40 Watts as is required to ablate tissue using the prior art probes.

The present embodiments do not require the same level of precaution as the prior art probes. The ease of use, particularly when the distal opening is maintained in contact with the target tissue eliminates much of the anxiety associated with use of traditional electrosurgical probes in the upper and lower gastrointestinal tracts, and in the bronchioles of lung transplant patients.

Although the present invention has been described with reference to the preferred embodiments illustrated in the accompanying drawings, it is to be understood that various modifications, additions and/or alterations may be made to the parts previously described without departing from the ambit of the present invention as defined in the claims appended hereto.

The word proximal as used herein refers to parts in closer proximity to the surgeon operating the probe and probe attachment. Following on from this, the word distal refers to parts located at a distance, relative to the surgeon.

The word surgeon as used herein is not intended to exclude other practitioners and users of the inventive probe and probe attachment. Thus, “surgeon” is intended to cover practitioners including but not limited to gastroenterologists, respiratory physicians and other personnel who are likely to use electrosurgery and endoscopic devices.

1. A probe for use in gas enhanced electrosurgery, the probe comprising:
(a) a lumen having a proximal end and a distal opening configured for contacting target tissue to achieve a treatment effect;
(b) an electrode within the lumen, the electrode having a proximal end connectable to a high frequency current source and a discharge end recessed within the distal end of the lumen; and
(c) a gas outlet in a distal region of the lumen for relieving lumen gas pressure;
wherein the distal opening of the lumen contacts target tissue during use while the electrode is activated, and excess gas pressure within the lumen is relieved through the gas outlet.

2. The probe of claim 1, wherein the distal opening is shaped to create a desired treatment effect in the target tissue.

3. The probe of claim 1, wherein the distal opening is oblique to facilitate treatment of a larger area of target tissue.

4. The probe of claim 3, wherein the gas outlet is positioned to minimise the simultaneous occlusion of the gas outlet and the distal opening with the target tissue.

5. The probe of claim 1, wherein the gas outlet is positioned adjacent to the discharge end of the electrode.

6. The probe of claim 1, wherein the gas outlet is positioned just distal of the discharge end of the electrode.

7. The probe of claim 1, wherein there is more than one gas outlet for relieving excess gas pressure within the lumen.

8. The probe of claim 1, wherein the distal region of the lumen includes a transparent portion.

9. The probe of claim 8, wherein the transparent portion is manufactured from an insulating material.

10. The probe of claim 8, wherein the transparent portion is ceramic.

11. The probe of claim 1, wherein the distal opening has a low coefficient of friction coating for suppressing eschar build up during use.

12. The probe of claim 1 further comprising a marking on the distal region of the lumen to indicate orientation of the distal opening when in use.

13. The probe of claim 1 further comprising a pivot means for enabling the distal opening to maintain contact with the target tissue during movement of the probe or target tissue.

14. The probe of claim 1, wherein gas within the lumen includes an ionizable gas.

15. The probe of claim 14, wherein the ionizable gas is selected from the group consisting of argon and helium.

16. The probe of claim 1, wherein a distal portion of the electrode is tungsten with a highly conductive coating at its working tip.

17. The probe of claim 16, wherein the highly conductive coating is silver.

18. The probe of claim 1 which is adapted for use within a working channel of a flexible endoscope.

19. The probe of claim 1, wherein the discharge end of the electrode is recessed within the distal end of the lumen to preclude contact between the discharge end and the target tissue.

20. A method of treating target tissue comprising, contacting the distal opening of the probe of claim 1 to the target tissue and drawing the distal opening along the target tissue surface during activation of the electrode.

21. An attachment for an electrosurgical probe having an electrode comprising:
(a) a lumen having a proximal end connectable to the probe and a distal opening being a working tip; and
(b) a gas outlet in the lumen for releasing lumen gas pressure; wherein the attachment is coupled to the probe, and wherein the electrode is recessed within the lumen which precludes direct contact with target tissue, and wherein the working tip contacts the target tissue during activation of the electrode to achieve a treatment effect, and excess gas pressure within the lumen is relieved through the gas outlet.

22. The attachment of claim 21, wherein the distal opening is shaped to create a desired treatment effect in the target tissue.

23. The attachment of claim 22, wherein the distal opening is oblique.

24. The attachment of claim 21, wherein the gas outlet is positioned to minimise the simultaneous occlusion of the gas outlet and the distal opening with target tissue.

25. The attachment of claim 21, wherein the gas outlet is positioned just distal of the discharge electrode.

26. The attachment of claim 21 further including a transparent window portion.

27. The attachment of claim 21, wherein the attachment is transparent.

28. The attachment of claim 21, wherein the attachment is ceramic.

29. The attachment of claim 21, wherein the distal opening has a low coefficient of friction coating to suppress eschar build up during use.

30. The attachment of claim 21 further comprising a marking on the outer surface of the lumen to indicate the orientation of the distal opening when in use.

31. The attachment of claim 21 further comprising a pivot means for enabling the distal opening to maintain contact with the target tissue during movement of the probe or target tissue.

32. The attachment of claim 21 which is coupled with a probe and inserted within a working channel of a flexible endoscope.

33. An endoscopic electrosurgery probe comprising, a flexible lumen with a proximal end connectable to a source of ionisable gas and a distal opening, an electrode having a proximal end attachable to a high frequency current source and a discharge tip recessed within a distal region of the lumen and positioned to preclude direct contact with target tissue, and wherein the lumen further comprises a gas outlet near the electrode discharge tip, and wherein the distal opening contacts target tissue while the electrode is activated by applying a high frequency current to the electrode, and shrouding the electrode in ionisable gas, creates a treatment effect in the target tissue at the gas-tissue interface, and wherein excess gas pressure within the lumen is relieved through the gas outlet.

34. The endoscopic electrosurgery probe of claim 33, wherein the treatment effect is ablation of the target tissue.

35. A probe for use in electrosurgery, the probe comprising:
(a) a lumen having a proximal end and a distal opening;
(b) an electrode within the lumen, the electrode having a proximal end connectable to a high frequency current source and a discharge end recessed within the lumen; and
(c) a gas outlet in a distal region of the lumen for releasing lumen gas pressure;
wherein during use, the distal opening of the lumen contacts target tissue while the electrode is activated, and gas pressure within the lumen is released through the gas outlet to preclude a gas embolism.
